(19) United States
(12) Patent Application Publication
(10) Pub. No.: US 2002/0142944 A1
(43) Pub. Date: Oct. 3, 2002

(54) Treatment of ocular disorders

(75) Inventors: Robertus Wilhelmus Aloysius Maria Kuijpers, Rotterdam (NL); Petrus Martinus van Hagen, Rotterdam (NL); Goitzen Seerp Baarsma, Rotterdam (Hilegersberg) (NL)

Correspondence Address: Ronald J. Baron, Esq., HOFFMANN & BARON. LLP, 6900 Jericho Turnpike, Syosset, NY, 11791, US

(73) Assignee: Erasmus Universiteit Rotterdam

(21) Appl. No.: 10/079,931

(22) Filed: Feb. 19, 2002

Related U.S. Application Data
(63) Continuation of application No. 09/258,240, filed on Feb. 26, 1999.

(51) Int. Cl. A61K 38/31

(52) U.S. Cl. 514/2

(57) ABSTRACT

The present invention relates to a method for treatment of an ocular disorder, and especially retinal edema, comprising the administration to a patient of a compound that binds to at least one somatostatin receptor.

### FIELD OF THE INVENTION

The invention relates to a method for treating ocular disorders, in particular to a method for treating retinal edema, and especially macular edema.

### BACKGROUND OF THE INVENTION

Retinal edema is identified as an abnormal accumulation of fluid in retinal cells. Macular edema is identified as intraretinal fluid in the macular region. This phenomenon is often a complication of a variety of diseases, including ocular diseases such as uveitis and may result in a decreased visual acuity. In fact, cystoid macular edema is the most important cause for visual impairment in uveitis.

Retinal edema evolves from leaking retinal vessels of a deficient aqueous pump function by the retinal pigment epithelium cells. In order to diminish the edema either the leakage should be stopped or the pump function should be regulated.

Retinal edema may result from a breakdown of the blood retinal barrier resulting in leakage from retinal capillaries or by a reduction of the active transport of fluid from the retina towards the choroid, or both. Clinically important sequelae of retinal, and in particular macular, edema are loss of visual acuity and secondary structural changes of the retinal anatomy with photoreceptor loss. Until now, the main approach in treatment of retinal edema is treatment of the underlying disease, when possible. Thus, immune suppressive therapy in uveitis may lead to inhibition of the inflammation and secondary to diminishing of macular edema. Symptomatic treatment of retinal edema includes treatement with various pharmaceuticals such as diclofenac eye drops, peribulbar injections of betamethasone, acetazolamide and enalapril and prostaglandin inhibitors. Although in a number of cases, there is a relief of the discomfort caused or even a cure of the edema, there is a need for an alternative method of treatment. In addition, there are types of retinal edema which do not respond to any of the known treatments, e.g. idiopathic cystoid macular edema.

It is an object of the present invention to find such a treatment.

It is a further object of the present invention to find new treatments for retinal edema that could not be treated successfully until now.

### SUMMARY OF THE INVENTION

In accordance to the present invention, it has been found that the objects of the invention can be achieved by the administration to a patient of compounds that bind to at least one somatostatin receptor, such as hSST-1, hSST 2, hSST-3, hSST-4 or hSST-5, and preferably to at least the hSST-2 receptor, and more preferably to the hSST-2a receptor.

### DETAILED DESCRIPTION OF THE INVENTION

In accordance with the present invention, it has been found that a beneficial effect is obtained when compounds that bind to at least one somatostatin receptor are administered to patients suffering from retinal edema. More in particular, if patients suffering from cystoid macular edema are treated with compounds that bind to somatostatin receptors, such as hSST-1, hSST-2, hSST-3, hSST-4 or hSST-5, it was found that the edema diminished or even disappeared. Further inflammation reactions decreased.

Preferably, said compound binds to somatostatin receptors in the nanomolar range.

In a preferred embodiment of the method of the invention, the said compound binds to a hSST-2 receptor, most preferably to a hSST-2a receptor.

The ocular disorders to be treated are generally caused by retinal edema, more in particular by macular edema, and particularly by cystoid macular edema (CME). Further it has been found that idiopathic CME also diminishes.

In accordance with the present invention it has been found that the somatostatin receptor binding compounds in the method of treatment of the present invention have a number of beneficial effects. These effects can be subdivided in three categories.

Category (1) is associated with the stopping of leakage in existing and new ocular vessels. This effect is relevant to e.g. the treatment of macular edema, accumulation of subretinal fluid, exudates in age related macular degeneration (AMD) and exudates in diabetic retinopathy (DR).

Category (2) deals with the restoration and/or regulation of retinal pigment epithelium function with respect to fluid and ion transport. Typical examples are selected from AMD exudates, DR exudates, central serous chorio-retinopathy (CSCR), macular edema and accumulation of subretinal fluid.

In Category (3) neovascularization in AMD and DR is inhibited. A typical example is selected from subretinal neovascularization.

Utility preference is given to category (1) and (2); even more preferred is category (1).

Accordingly, the present invention relates to a method of treating an ocular disorder of category (1), (2) and (3), which method comprises the administration of a somatostatin analogue to a patient.

Suitable compounds to be used in the method of the present invention belong to the naturally occurring class of the somatostatins. Somatostatin is a neuropeptide which constitutes a multi gene peptide family with two principal bioactive products, somatostatin-14 and somatostatin-28. It acts on multiple organs including the brain gut, endocrine glands, pancreas, kidneys and the immune system (Reichlin S. Somatostatin (first of two parts). N Engl J Med 1983; 309:1495-501, Reichin S. Somatostatin (second of two parts). N Engl J Med 1983; 309:15556-63, Krulich I, Dhariwal A P, McCann S M. Stimulatory and inhibitory effects of purified hypothalamic extracts on growth hormone release from rat pituitary in vitro. Endocrinology 1968; 83:783-90, Brazeau P, Vale W, Burgus R, Lang N, Butcher M, Rivier J, Guillemin R. Hypothalamic polypeptide that inhibits the secretion of immunoreactive pituitary growth hormone. Science 1973; 179:77 9, Lucey M R. Endogenous somatostatin and the gut. Gut 1986;27:457-67, MehlerP S, Sussman A L, Maman A, Leitner J W, Sussman K E. Role of insulin secretagogues in the regulation of somatostatin binding by isolated rat islets. J Clin Invest 1980;66:1334-8). Somatostatin binds to 5 types of G-protein coupled transmembrane receptors (sst) Schönbrunn A, Tashjian H Jr. Characterization of functional receptors for somatostatin in ra pituitary cells in culture. J Biol Chem 1978; 253:6473-83. Reubi J C, Kvols L K, Krenning E P, Lamberts S W J. Distribution of somatostatin receptors in normal and tumor tissue. Metabolism 1990; 39 Suppl 2:78-81, Patel Y C, Greenwood M T, warszynska A, Panetta R, Strikant C B. All five cloned human somatostatin receptors (hSSTR1-5) are functionally coupled to adenylyl cyclase. Biochem Biophys Res Commun 1994;198:605-12). Somatostatin analogues can be used as well.

More in general, the somatostatin class is a known class of small peptides comprising the naturally occurring somatostatin-14 and analogues having somatostatin related activity, e.g. as disclosed by A. S. Dutta in Small Peptides, Vol. 19, Elsevier (1993). By the term a “somatostatin peptide” or “a somatostatin analogue” as used herein is meant any straight or cyclic polypeptide having a structure based on that of the naturally occurring somatostatin-14 wherein one or more amino acid units have been omitted and/or replaced by one or more other amino radical(s) and/or wherein one or more functional groups have been replaced by one or more other functional groups and/or one or more groups have been replaced by one or several other isosteric groups. In general, the term covers all modified derivatives of the native somatostatin which exhibit a somatostatin related activity e.g., that bind to at least one somatostatin receptor (hSST-1, hSST-2, hSST-3, hSST-4 or hSST-5), preferably to at least the hSST-2 receptor.

The terms a somatostatin, a somatostatin peptide and a somatostatin analogue are used within this disclosure as synonyms.

Cyclic, bridged cyclic and straight-chain somatostatin analogues or derivatives are known and have been described together with processes for their production e.g. in U.S. Pat. No. 4,310,518, U.S. Pat. No. 4,235,886 and EP-A-0 001 296, the contents thereof, in particular with respect to the compounds, being incorporated herein by reference.

Preferred somatostatin analogues are e.g. compounds of formula (I).

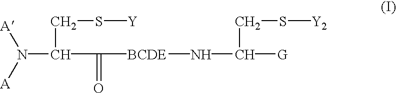

wherein

A is C₁₋₁₂alkyl, C₇₋₁₀phenylalkyl or a group of formula RCO—, whereby

(i) R is hydrogen, C₁₋₁₁alkyl, phenyl or C₇₋₁₀phenylalkyl, or

(ii) RCO— is

a) a D-phenylalanine residue optionally ring-substituted by halogen NO₂, NH₂, OH, C₁₋₃alkyl and/or C₁₋₃alkoxy; or

b) the residue of a natural or a synthetic α-amino-acid other than defined under a) above, or of a corresponding D amino acid, or

c) a dipeptide residue in which the individual amino acid residues are the same or different and are selected from those defined under a) and/or b) above,

the α-amino group of amino acid residues a) and b) and the N-terminal amino group of dipeptide residues c) being optionally mono- or di-C₂₋₁₃alkylated or substituted by C₁₋₃alkanoyl;

A is hydrogen or C₁₋₃alkyl,

Y₁ and Y₂ represent together a direct bond or each of the Y₁ and Y₂ is hydrogen

B is -Phe- optionally ring-substituted by halogen, NO₂, NH₂, OH, C₁₋₃alkyl and/or C₁₋₃alkoxy (including pentafluoroalanine), naphthylalanine or pyridylalanine,

C is (L)-Trp- or (D)-Trp- optionally a-N-methylated and optionally benzene-ring-substituted by halogen, NO₂, NH₂, OH C₁₋₃alkyl and/or C₁₋₃alkoxy,

D is Lys, 4-aminocyclohexylAla or 4-aminocyclohexylGly

E is Tnr, Ser, Val, Tyr, ILe, Leu or an aminobutyric or aminoisobutyric acid residue

G is a group of formula: —COOR₇, —CH₂OR₁₀, —CONR₁₁R₁₂ or

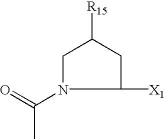

wherein

R₇ is hydrogen or C₁₋₃alkyl,

R₁₀ is hydrogen or the residue or a physiologically acceptable, physiologically hydrolysable ester,

R₁₁ is hydrogen, C₁₋₃alkyl, phenyl or C₃₋₁₀phenyl-alkyl,

R₁₂ is hydrogen, C₁₋₃alkyl or a group of formula —CH (R₁₃)—X₁,

R₁₃ is CH₂OH, —(CH₂)2-OH, —(CH₂)₃—OH, or —CH (CH₃) OH or represents the substituent attached to the a-carbon atom of a natural or synthetic a-amino acid (including hydrogen) and

X₁ is a group of formula —COOR₇, —CH₂OR₁₀ or —CO—NR₁₄R₁₃

wherein

R_{7 and R}₁₀ have the meanings given above,

R₁₄ is hydrogen or C₁₋₃alkyl and

R₁₅ is hydrogen, C₁₋₃alkyl, phenyl or C₇₋₁₀phenylalkyl, and

R₁₆ is hydrogen or hydroxy,

with the proviso that

when R₁₂ is —CH (R₁₅)—X₁ then R₁₂ hydrogen or methyl,

wherein the residues B, D and E have the L-configuration, and the residues in the 2- and 7-position each independently have the (L)- or (D)-configuration,

in free form or in pharmaceutically acceptable salt or complex form.

Individual compounds of formula I suitable in accordance with the present invention are the following somatostatin analogues:

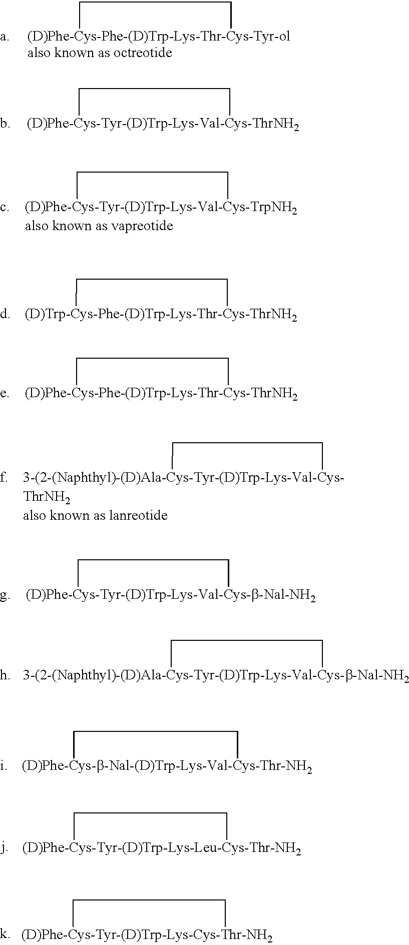

More preferred compounds of formula (I) are compounds (a)-(k).

A highly preferred Compound of formula (I) is octreotide.

Compounds of formula (I) may exist e.g. in free from, salt form or in the form of complexes thereof. Acid addition salts may be formed with e.g. organic acids, polymeric acids and inorganic acids. Such acid addition salt forms include e.g. the hydrochlorides and acetates. Complexes are e.g. formed from compounds of the invention on addition of inorganic substances, e.g. inorganic salts or hydroxides such as Ca- and Za-salts, and/or and addition of polymeric organic substances.

According to the invention, the compound binding to somatostatin receptor is preferably administered in the form of a pharmaceutical composition, by any conventional route, in particular enterally, e.g. orally, e.g. in the form of tablets, capsules, drink solutions, emulsions or microemulsion preconcentrates, nasally, pulmonary (by inhalation), parenterally, e.g. in the form of injectable solutions or suspensions, or topically. The compound is preferably administered parenterally, typically subcutaneously, e.g. by injection and/or infusion

In a further aspect, the compound capable of binding to a somatostatin is administered topically to an individual, typically in the form of an ophthalmic liquid preparation (eye drop), in the form of a gel and/or in the form of an ointment.

The compound capable of binding to a somatostatin receptor may also be administered locally e.g. intravitreally and peribulbarely.

The amount administered is determined taking into account various factors such as the etiology and severity of the desease, and the patient's condition. A somatostatin analogue may be administered, e.g. subcutaneously in a dosage range of about 100 μg to 10 mg per day as a single dose or in divided doses. Thus octreotide may be administered at a dose of from 0.2 mg to 10 mg twice or three times daily. When administered as a slow release form, such formulation may comprise the somatostatin peptide in a concentration from 2.0 to 10% by weight. The release period of such a formulation may be from 1 week to about 2 months.

### THE BEST MODE

The best results up to now have been obtained by using octreotide as the somatostatin receptor binding compound. These results are elaborated herein-below:

The present invention will be further illustrated by the following non-limiting examples.

### EXAMPLES

Treatment of cystoid macular edema

A 21 year old man was found to have moderate color-vision defects at routine examination. One year later, he noticed blurred vision. His visual acuity was 20/50 in the right eye and 20/40 in the left eye (by Snellen chart). Because he had bilateral cystoid macular edema, he was treated with diclofenac eye drops, peribulbar injections of betamehtasone, acetazolamide, and enalapril without beneficial effect on the edema or visual acuity. His visual acuity slowly deteriorated in the subsequent years. When we examined the patient three years after he first noted blurring of his vision, his visual acuity was 20/100 in each eye, the macular regions showed large cystoid lesions and there was no intraocular inflammation (anterior chamber flare or cells, vireous cells, vascular sheathing, exudates, or pare planitis). He had no family history of macular edema. Fluorescein angiography showed accumulation of dye in the cystoid lesions. Because the previous therapy had failed to correct the problem, the patient was treated with octreotide 100 μg subcutaneously three times daily, after he gave informed consent. He noted visual improvement after six weeks, and when tested after eight weeks his visual acuity was 20/40 in the right eye and 20/50 in the left eye. Ophthalmoscopically, the cysts had dried. The injections were stopped four weeks later, because there was no further improvement.

Within two weeks the patient noticed increased reading difficulty, his visual acuity was 10/50 in each eye, and some fluid was observed ophthalmoscopically. Treatment with 100 μg or octreotide subcutaneously once daily was resumed. Three months later the patient's visual acuity was 20/40 in each eye; fluorascein angiography showed some foveal fluorascein leakage. He stopped treatment a second time; after four weeks his visual acuity was 20/70 in the right eye and 20/50 in the left eye. Octreotide therapy was resumed, and four weeks later his visual acuity was 20/40 in each eye. The patient had no side effects from the treatment. The decreased macular edema during treatment with octreotide, the recurrence after stopping treatment, and the response after restarting it suggest that the changes were due to octreotide.

### Example 2

Patients were treated in accordance to the declaration of Helsinki. In this study, 10 consecutive patients from the Department of Ophthalmology, Erasmus University Medical Center, and the Eye Hospital Rotterdam, were selected because of refractory CME due to chronic uveitis. Since CME in uveitis can be influenced in time by multiple factors, only patients suffering from macular edema for more than 6 months were included, regardless of their immune suppressive treatment. Six patients had bilateral CME and four patients unilateral. Three patients with pars planitis needed no treatment for their inflammation but still had marked CME. Fluorescein angiography was performed within one week before starting octreotide treatment. Octreotide therapy was given after informed consent had been obtained. Dosage was started with 100 μg subcutaneously, on the first day, two times 100 μg on the second day and three times 100 μg from the third day onwards. When possible, the long-acting repeatable (LAR) formulation was prescribed in a dosage of 20 mg per 4 weeks intramuscularly, which became available during the studies. The dose of patient 3, a 15 year old boy, was adjusted for his age to 10 mg per month (im). Immune suppressive treatment was adjusted as based on inflammatory activity. Visual acuity was measured after 2, 4, and 12 weeks of treatment. Fluorescein angiography was repeated after three months of treatment.

Clinical effects of octreotide

All patients concluded three months therapy. In none of the patients visual acuity or inflammation activity deteriorated. Four patients showed a decreased inflammation, leading to a tapered immunosuppressive therapy.

By stereoscopic examination the CME had dissolved in 5 eyes and diminished in a further 11 eyes. Fluorescien angiographic macular edema was clearly diminished in only 3 eyes, was diminished but still visible in 9 eyes, and was unaltered in 3 eyes, in one eye the angiogram could not be intrepreted because of poor quality. No side effects were observed, except diarrhoea during the first days of treatment in 4 patients. Ocular hypertension was measured in one patient at week 4, which was treated with timolol.

### Example 3

Immuno histopathology

For histopathology 3 human eyes were obtained after enucleation. Two eyes were enucleated because of malignant melanoma and one because of a painfull blind eye with corneal ulceration and perforation, showing extensive CME. In order to investigate the expression of somatostatin receptors, immunohistochemical analysis was performed with rabbit polyclonal anti-somatostatin receptor subtype -1 and -2a antibodies (anti-sst₁ and anti-sst_{2A}) (Dournaud P, Cu Y Z, Schönbrunn A, Mazella J, Tannenbaum G S, beauder A. Localization of the somatostatin receptor sst2A in rat brain using a specific antipeptide antibody. J Neurosci 1996; 16:4468-78). Specifically of the antibodies has been demonstrated before by Western Blot analysis. Frozen tissue sections (5 μm) mounted on uncoated glass slides were dried, fixed in 10% formalin, rinsed in PBS and preincubated for 15 min. at room temperature (RT) with 10% normal goat serum in PBS/5%BSA. Incubation with anti-sst₁ and anti-sst_{2A} antibodies (dilution 1:1000) was carried out overnight at 4° C. The sections were rinsed twice in PBS and incubated for 30 min. at RT with alkaline phosphatase-conjugated goat-anti-rabbit immunoglobulin (GαRig-AP, D0487, Dakopatts, Glostrup, Denmark) diluted 1:50 in PBS/5%BSA containing 2% normal human serum. Hereafter, the sections were rinsed twice with PBS. Alkaline phosphatase activity was revealed by new fuchcine as the chromogen in the presence of levamisole to block endogenous alkaline phophatase activity, followed by hematoxylin staining. Controls for immunohistochemistry included: 1) omission of the primary antibody, 2) incubation with normal rabbit serum, 3) pre-absorption of the antiserum with the immunizing peptide.

Immunohistochemistry

The polyclonal antibodies against sst₁ and sst_{2A} are specific for human somatostatin receptor 1 and 2a respectively. A red chromogen was used to differentiate from the brown pigment in the retinal pigment epithelium (RPE).

Sst₁ positive staining was noted in ganglion cells and amacrine cells of the inner nuclear layer, and the plexiform layer, the inner and outer nuclear layer, and the apical membranous part of the retinal pigment epithelium. In the retina with CME similar staining for sst_{2A} was observed.

1. A method for treatment of an ocular disorder, comprising the administration to a patient of a compound that binds to at least one somatostatin receptor.

2. The method of claim 1, wherein the said compound binds to a hSST-2 receptor.

3. The method of claim 1, wherein said ocular disorder is caused by retinal edema.

4. The method of claim 3, wherein said ocular disorder is caused by macular edema.

5. The method of claim 1, wherein said compound is selected from the group consisting of the somatostatins and somatostatin analogues.

6. The method of claim 5, wherein said compound is octreotide.

7. The method of claim 1, wherein said compound is administered topically.

8. The method of claim 7, wherein said compound is administered in the form selected from the group consisting of eye drops, eye gel and eye ointment.

9. The method of claim 1, wherein said compound is administered subcutaneously.
